(19) United States
(12) Patent Application Publication
(10) Pub. No.: US 2011/0313287 A1
(43) Pub. Date: Dec. 22, 2011

(54) CHEMICAL LIQUID INJECTOR AND X-RAY CT SYSTEM

(75) Inventors: Sei Komatsu, Hyogo (JP); Shigeru Nemoto, Tokyo (JP)

(73) Assignee: Nemoto Kyorindo Co., Ltd., Tokyo (JP)

(21) Appl. No.: 13/254,829

(22) PCT Filed: Mar. 3, 2010
(86) PCT No.: PCT/JP2010/053440

§ 371 Date: Sep. 2, 2011

(30) Foreign Application Priority Data
Mar. 4, 2009 (JP) 2009-051068

(51) Int. Cl. A61B 6/00; A61M 5/31

(52) U.S. Cl. 600/432

(57) ABSTRACT

Chemical liquid injector 100 has injection head 110 on which a syringe filled with a chemical liquid is mounted and injection control unit 101. Injection control unit 101 has input device 142 receiving the input of data including the maximum CT value provided in test injection, a tube voltage value to be applied to X-ray tube 311 of X-ray CT apparatus 300 in main injection, and a targeted CT value in the main injection, injection amount computing section 146 calculating the amount of contrast medium to be injected in the main injection by using those data items, and control section 141 controlling the operation of injection head 110 in the main injection such that the calculated amount of contrast medium is injected.

### TECHNICAL FIELD

The present invention relates to a chemical liquid injector for use in injecting a contrast medium into a patient in order to obtain CT images of the patient, and an X-ray CT system having the chemical liquid injector and an X-ray CT apparatus.

### BACKGROUND ART

Currently employed medical imaging diagnostic apparatuses include CT (Computed Tomography) scanners, MRI (Magnetic Resonance Imaging) apparatuses, PET (Positron Emission Tomography) apparatuses, angiography apparatuses, MRA (MR Angiography) apparatuses and the like. For obtaining CT images of a patient with the abovementioned apparatuses, a chemical liquid such as a contrast medium or physiological saline is often injected into the patient's body.

In general, the injection of the chemical liquid into the patient is automatically performed by using a chemical liquid injector. The chemical liquid injector has an injection head on which a syringe filled with the chemical liquid is removably mounted and an injection control unit which controls the operation of the injection head. The syringe has a cylinder and a piston inserted thereinto to be movable in its axis direction. The chemical liquid is filled in the cylinder. The injection head includes a piston driving mechanism for pushing the piston of the syringe mounted on the injection head into the cylinder. After an injection needle is connected to the end of the cylinder through an extension tube and the injection needle is inserted into a patient's blood vessel, the piston driving mechanism pushes the piston into the cylinder, so that the chemical liquid in the cylinder can be injected into the patient.

In obtaining CT images with the contrast medium, the amount of contrast medium to be injected is set so as to provide favorable contrast enhancement effects. The obtaining of CT images is desirably performed at the time when the contrast medium reaches a region to be imaged or imaging region after the contrast medium is injected and flows on the bloodstream of the patient. Thus, in a conventional imaging diagnostic apparatus, monitoring scan of the imaging region may be performed prior to main scan in order to measure the time taken for the contrast medium to reach the imaging region. The measurement of the time taken for the contrast medium to reach the imaging region is performed by using a CT value which is proportional to the concentration of the contrast medium in blood. For example, Patent Document 1 has disclosed a CT apparatus which performs monitoring scan to calculate a CT value at an imaging region after the injection of a contrast medium into a patient, and performs main scan when predetermined conditions are satisfied, such as when the CT value exceeds a preset threshold.

### PRIOR ART REFERENCE

### Patent Document

Patent Document 1: Japanese Patent Laid-Open No. 2007-111255

### SUMMARY OF THE INVENTION

### Problems to be Solved by the Invention

Typically, the amount of contrast medium to be injected is often determined in accordance with the weight of a patient, and the same amount of contrast medium is injected into patients of the same weight. In reality, however, factors other than the weight have an influence on the contrast enhancement effect. Even when the same amount of contrast medium is injected into patients of the same weight, the resulting CT values vary greatly among the patients and it is difficult to obtain the stable contrast enhancement effect among the patients. To obtain the stable contrast enhancement effect, factors other than the weight need to be considered in determining the amount of contrast medium. However, this requires the measurements of the other factors as required and the input of them as data in order to determine the amount of contrast medium, thereby complicating the process for determining the amount of contrast medium.

The amount of contrast medium to be injected is often set on the premise that an X-ray CT apparatus aims to be able to capture images with the highest possible resolution. However, clear images in all parts may not be needed depending on the object of diagnosis, and conventionally, the amount of contrast medium is not necessarily set in accordance with the object of diagnosis. In addition, the injection of the contrast medium into the patient affects the patient's body, so that the smallest possible amount is required.

It is an object of the present invention to allow the easy determination of the amount of contrast medium in accordance with individual differences and to enable the capture of images having information necessary for diagnosis with a smaller amount of contrast medium in a chemical liquid injector for injecting the contrast medium in order to obtain CT images in a CT apparatus.

### Means for Solving the Problems

To achieve the abovementioned object, the present invention provides a chemical liquid injector comprising an injection head injecting a contrast medium into a patient through operation of a syringe filled with the contrast medium and a control section controlling operation of the injection head so as to perform test injection prior to main injection, for obtaining CT images of the patient with an X-ray CT apparatus,

characterized in that the chemical liquid injector further comprises:

input means for receiving input of data including a maximum CT value measured by the X-ray CT apparatus in the test injection, a tube voltage value to be applied to an X-ray tube of the X-ray CT apparatus in the main injection, and a target CT value serving as a targeted CT value in the main injection; and

an injection amount computing section calculating an amount of contrast medium to be injected in the main injection based on the maximum CT value and the tube voltage value such that a maximum CT value measured in the main injection is the target CT value,

wherein the control section controls the injection head in the main injection such that the amount of contrast medium calculated in the injection amount computing section is injected from the syringe.

The present invention also provides an X-ray CT system including the chemical liquid injector according to the present invention and an X-ray CT apparatus, wherein the chemical liquid injector and the X-ray CT apparatus can transmit and receive data therebetween through interfaces, and the maximum CT value measured by the X-ray CT apparatus in the test injection is transmitted to the injection amount computing section through the interface.

### Effect of the Invention

According to the present invention, it is possible to determine simply the optimal amount of contrast medium in accordance with individual differences and to capture images having information necessary for diagnosis with a smaller amount of contrast medium.

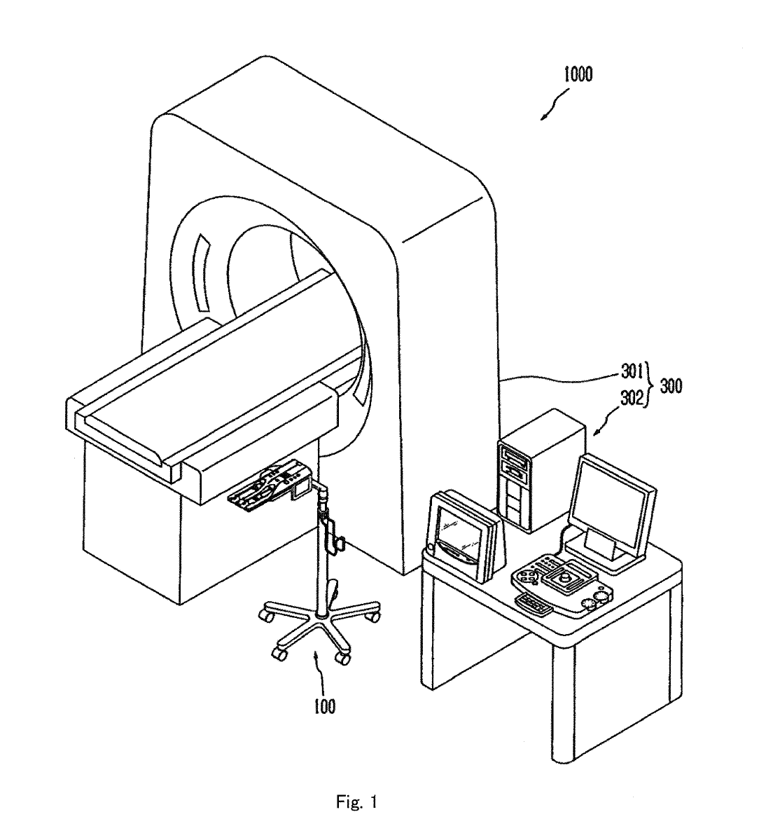

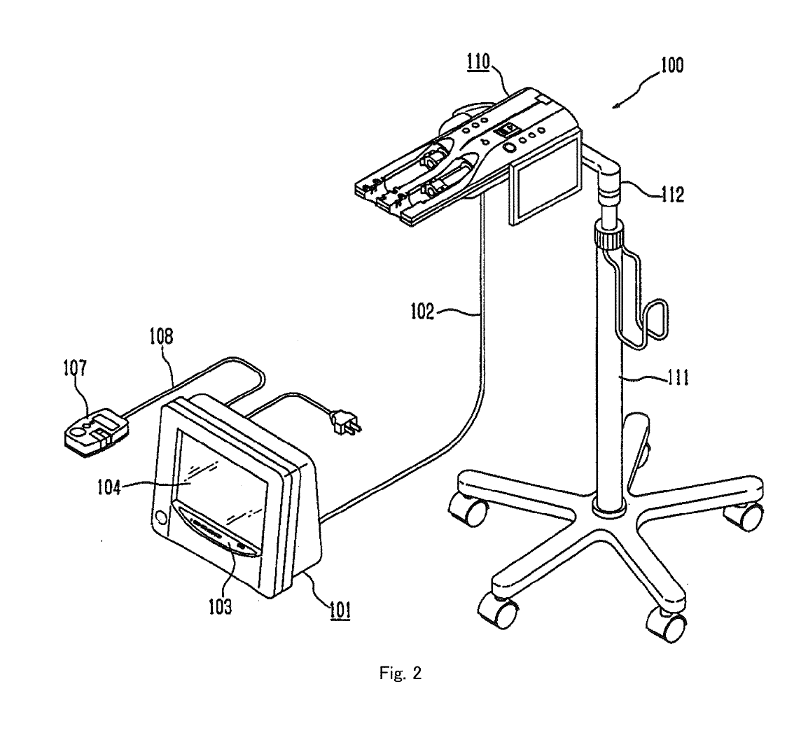

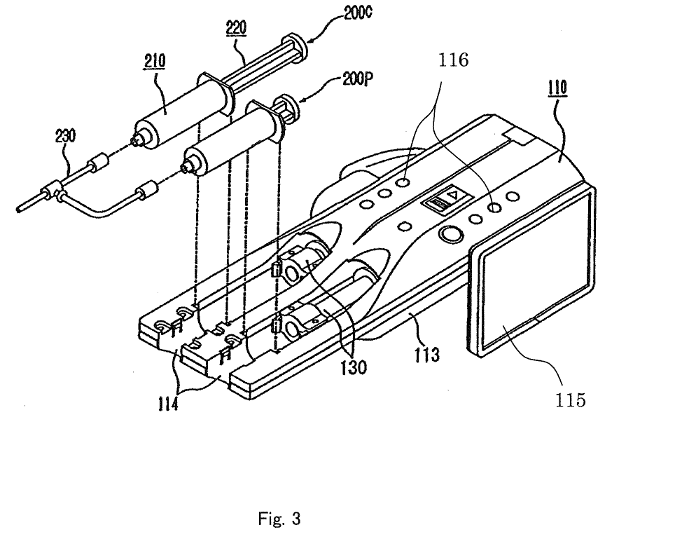

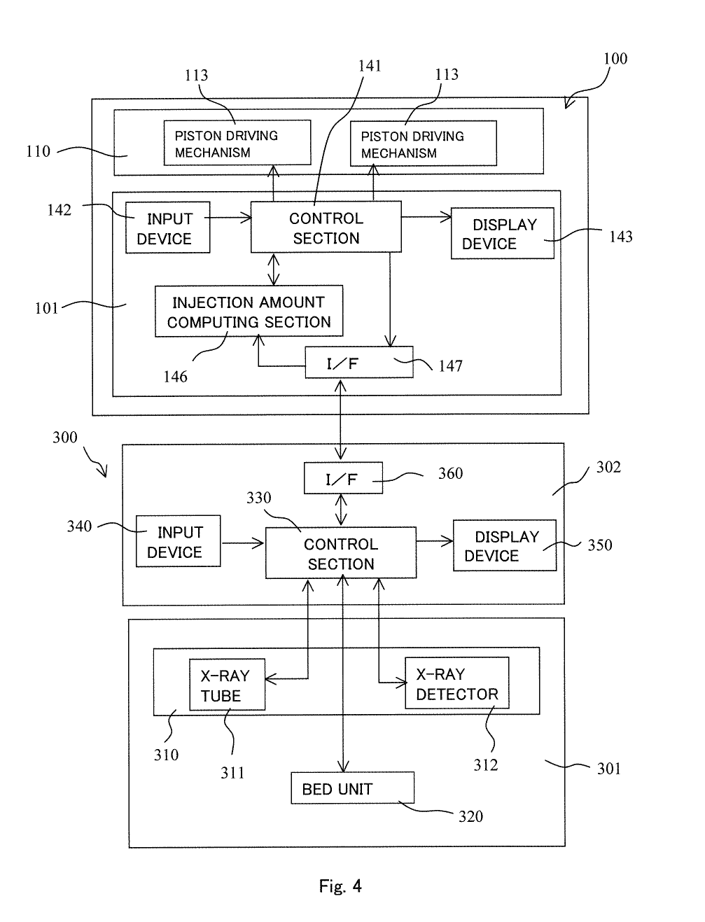

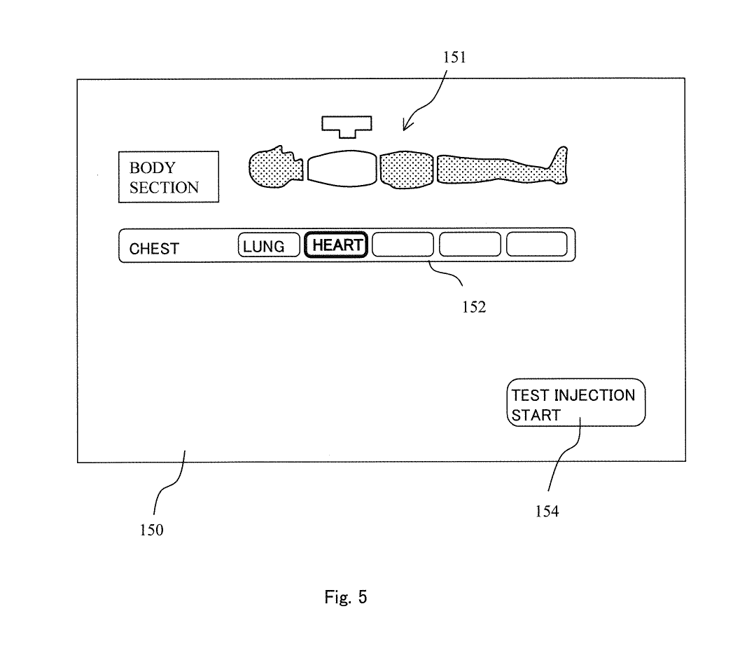

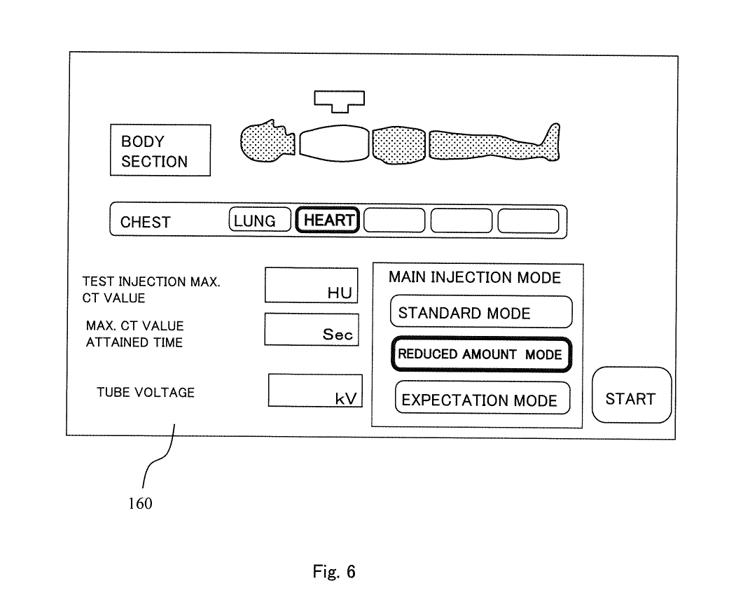

### BRIEF DESCRIPTION OF THE DRAWINGS

[FIG. 1] A perspective view showing an X-ray CT system according to an embodiment of the present invention.

[FIG. 2] A perspective view showing a chemical liquid injector shown in FIG. 1.

[FIG. 3] A perspective view showing an injection head shown in FIG. 2 together with syringes mounted thereon.

[FIG. 4] A block diagram in which the X-ray CT system shown in FIG. 1 is functionally represented.

[FIG. 5] A diagram showing an example of a setting screen for test injection.

[FIG. 6] A diagram showing an example of a setting screen for main injection.

### EMBODIMENT FOR CARRYING OUT THE INVENTION

Next, an embodiment of the present invention will be described with reference to the drawings.

Referring to FIG. 1, X-ray CT system 1000 according to an embodiment of the present invention, which has chemical liquid injector 100 and X-ray CT apparatus 300 serving as an imaging diagnostic apparatus, is shown. X-ray CT apparatus 300 and chemical liquid injector 100 are connected to each other such that data about chemical liquid injection performed in chemical liquid injector 100 can be transmitted to X-ray CT apparatus 300.

X-ray CT apparatus 300 has scanner 301 which performs imaging operation and imaging control unit 302 which controls the operation of scanner 301. While FIG. 1 shows all of the components of X-ray CT scanner 1000 placed in one room, imaging control unit 302 is placed in a different room from that of scanner 301 and chemical liquid injector 100 in actually obtaining CT images of a patient.

For example as shown in FIG. 2, chemical liquid injector 100 has injection head 110 attached to the top end of stand 111 by movable arm 112 and injection control unit 101 connected to injection head 110 through cable 102. Injection control unit 101 includes main operation panel 103, touch panel 104 serving as display means and input means, hand unit 107 connected to a main body of injection control unit 101 through cable 108 and serving as auxiliary input means, and the like.

As shown in FIG. 3, injection head 110 has two concave portions 114 formed as a syringe holding portion in an upper surface of head body 113. Syringes 200C and 200P are removably mounted in concave portions 114. Each of syringes 200C and 200P has cylinder 210 and piston 220. Each of syringes 200C and 200P is filled with a chemical liquid. For example, one syringe 200C can be filled with a contrast medium which can be injected to change the CT value of a patient and the other syringe 200P can be filled with physiological saline. Connection tube 230 joining into one at an intermediate portion and provided with an injection needle at its leading end is connected to the leading ends of two syringes 200C and 200P mounted on head body 113.

Two piston driving mechanisms 130 individually driven for operating pistons 220 of syringes 200C and 200P mounted in concave portions 114 are provided in association with concave portions 114 on injection head 110. Piston driving mechanisms 130 operate pistons 220 of syringes 200C and 200P mounted in concave portions 114 to allow the injection of the contrast medium and the physiological saline filled in syringes 200C and 200P, respectively, into the patient separately or simultaneously.

Each of a mechanism for holding syringes 200C and 200P on head body 113 and piston driving mechanism 130 in injection head 110 can be realized by employing a known mechanism typically used in the injector of this type.

FIG. 4 shows a block diagram in which X-ray CT system 1000 according to the present embodiment is functionally represented. Each block shown in FIG. 4 exists as at least a portion or at least a combination of portions of the configuration described in FIGS. 1 to 3 and may be configured as hardware or as a logical circuit.

In FIG. 4, X-ray CT apparatus 300 has scanner 301 and imaging control unit 302 as described above. Scanner 301 has bed unit 320 and imaging unit 310 which obtains X-ray CT images of a patient lying on his back on bed 320. Imaging unit 310 includes X-ray tube 311 which irradiates the patient lying on his back on bed unit 320 with X rays and X-ray detector 312 placed opposite to X-ray tube 311 so as to detect the X rays passed through the patient.

Imaging control unit 302 has control section 330, input device 340, display device 350 and interface (I/F) 360 with chemical liquid injector 100. Control section 330 controls the operation of scanner 301 based on data about imaging conditions transmitted from input device 340 or from the chemical liquid injector through interface 360 to take the data of the passed X-rays of the patient. Control section 330 also reconfigures images and displays diagnostic images on display device 350 based on the taken passed X-ray data, and also displays, on display device 350, data such as the maximum CT value and the time (maximum CT-value attained time) from the injection of the contrast medium to the attainment of the maximum CT value. A voltage (tube voltage) applied to X-ray tube 311 is typically 120 kV but can be changed in accordance with the adjustment by an operator or a command from control section 330 based on the input from chemical liquid injector 100 through interface 360.

On the other hand, in chemical liquid injector 100, injection control unit 101 has control section 141, input device 142, display device 143, injection amount computing section 146, and interface (I/F) 147.

Chemical liquid injector 100 has two injection modes of test injection and main injection as injection modes of the chemical liquid. The main injection is the injection of the chemical liquid for obtaining the CT images of the patient with the aim of performing a diagnosis, whereas the test injection is the injection of the contrast medium in which a smaller amount of contrast medium than that in the main injection is injected prior to the main injection to cause X-ray CT apparatus 300 to measure the maximum CT value as part of parameters for determining the amount of contrast medium to be injected in the main injection.

Input device 142 corresponds to main operation panel 103 and touch panel 104 shown in FIG. 2 and receives the input of data necessary for various setting of chemical liquid injector 100 by the operator and for the determination of the injection conditions of the chemical liquid. Display device 143 corresponds to touch panel 104 shown in FIG. 2 and displays a screen for setting chemical liquid injector 100, a screen for data input, a screen for showing the operational state, and the like. As described above, touch panel 104 has both of the function as part of input device 142 and the function of display device 143 in the present embodiment.

Injection amount computing section 146 calculates the amount of contrast medium that allows a target CT value, which is a targeted CT value in the main injection, to be obtained as the maximum CT value measured in the main injection, based on the maximum CT value measured in the test injection and the voltage value (tube voltage value) to be applied to X-ray tube 311 of X-ray CT apparatus 300 during the main injection.

To provide the maximum CT value, the value obtained in X-ray CT apparatus 300 through the test injection can be input from input device 142 by the operator and then input to injection amount computing section 146 through control section 141, or can be input automatically from X-ray CT apparatus 300 to injection amount computing section 146 when chemical liquid injector 100 and X-ray CT apparatus 300 are connected to each other through interfaces 147 and 360 capable of passing data bidirectionally as shown in FIG. 4. The tube voltage value for X-ray tube 311 and the target CT value are input from input device 142 by the operator and then input to injection amount computing section 146 through control section 141.

When the contrast medium is injected for the same time period, the amount of injected contrast medium and the rise in maximum CT value have a generally proportional relationship. Thus, the proportional relationship can be used to determine simply the amount of contrast medium to be injected in the main injection based on the amount of contrast medium injected in the test injection, the maximum CT value, and the target CT value.

As the tube voltage for X-ray tube 311 is lower, the generated X rays have a longer wavelength, the transmission force is lower, and the CT image has a higher contrast. That is, the tube voltage and the maximum CT value have a correlation, and lowering the tube voltage increases the maximum CT value. Thus, lowering the tube voltage can achieve a desired maximum CT value with a smaller amount of contrast medium. However, lowering the tube voltage tends to reduce the quality of the resulting CT image, so that the tube voltage is set in accordance with the object of imaging.

Injection amount computing section 146 calculates the amount of contrast medium to be injected in the main injection from a function preset by using the maximum CT value in the test injection, the tube voltage value for X-ray tube 311, and the target CT value as parameters, based on the relationship between the maximum CT value, the amount of contrast medium to be injected, and the tube voltage.

Control section 141 controls each portion of injection control unit 101 and injection head 110 such that the overall operation of chemical liquid injector 100 including the test injection and the main injection is performed in accordance with a sequence including an operation procedure, later described. Control section 141 is connected to X-ray CT apparatus 300 through interface 147 and transmits an injection start signal for contrast medium to X-ray CT apparatus 300 simultaneously with the start of the test injection and simultaneously with the start of the main injection.

Input device 142 and display device 143 can be provided not only for injection control unit 101 but also for injection head 110. In the present embodiment, injection head 110 includes a plurality of operation buttons 116 as part of input device 142 and includes display panel 115 as part of display device 143.

Next, the operation of the abovementioned X-ray CT system will be described.

First, the operator mounts syringe 200C filled with the contrast medium and syringe 200P filled with the physiological saline in predetermined concave portions 114 of injection head 110 and turns on the power of chemical liquid injector 100. When the power of chemical liquid injector 100 is turned on, control section 141 moves forward each of piston driving mechanisms 130 located at the most rearward position. This causes the trailing ends of pistons 220 of syringes 200C and 200P to be held by piston driving mechanisms 130. The injection needle has been connected to each of syringes 200C and 200P through extension tube 230 and inserted in a patient's blood vessel.

Next, control section 141 displays a setting screen for imaging region selection on display device 143. Description is made with the case where display device 143 is the touch panel and doubles as input device 142. Thus, in the following description, display device 143 and input device 142 are referred to as the touch panel in some cases.

FIG. 5 shows an example of the setting screen for imaging region selection displayed on display device 143. Body section selection button group 151, imaging region selection button group 152, and test injection start button 154 are displayed on setting screen 150 shown in FIG. 5.

Body section selection button group 151 has four buttons provided by dividing an image representing a lying patient into a head part, a chest part, an abdomen part, and a leg part. When the operator selects and touches one of them, the part corresponding to the touched button is input. The selected button is highlighted. FIG. 5 shows the state in which the chest part is selected.

Imaging region selection button group 152 has one or more buttons specifically showing imaging regions within the selected body section displayed in response to the input of the body section. When the operator selects and touches the button representing the intended imaging region, the imaging region corresponding to the touched region is input. FIG. 5 shows the state in which a heart part is selected as the imaging region.

The amount of contrast medium injected in the test injection is previously determined at the smallest possible level in the range in which the contrast enhancement effect can be obtained such that the burden on the patient due to the test injection is reduced. Specifically, the amount of contrast medium in the test injection ranges from 5 to 15 ml, preferably 5 to 10 ml.

After the selection of the imaging region is finished as described above, the operator touches test injection start button 154. This causes chemical liquid injector 100 to start the test injection by control section 141 moving piston driving mechanism 130 forward. A protocol for injecting the contrast medium at a certain injection speed during a predetermined injection time is set in control section 141 as an injection protocol of contrast medium. The amount of contrast medium to be injected is proportional to the moving distance of piston driving mechanism 130. Thus, control section 141 moves piston driving mechanism 130 forward at a certain speed by a distance in accordance with the amount determined for the test injection during the predetermined injection time. The injection speed of the contrast medium can be preset on the basis of the imaging region, for example, and may be changed by the operator as appropriate.

Simultaneously with the start of the test injection, a test injection start signal is transmitted from control section 141 to X-ray CT apparatus 300 through interface 147. X-ray CT apparatus 300 performs the monitoring scan a predetermined time after the start of the test injection. During the monitoring scan, X-ray CT apparatus 300 measures the CT value at regular time intervals, and the results are displayed on display device 350 as a graph, numeric values, images or the like.

When the monitoring scan is finished, X-ray CT apparatus 300 transmits a monitoring scan finish signal to chemical liquid injector 100 through interface 360. In chemical liquid injector 100, the monitoring scan finish signal is transmitted to control section 141 through interface 147. Upon reception of the monitoring scan finish signal, control section 141 displays an main injection setting screen on the touch panel for inputting conditions or the like necessary to cause injection amount computing section 146 to calculate the amount of contrast medium to be injected in the main injection. As described above, the amount of contrast medium to be injected in the main injection is determined by using the maximum CT value obtained in the test injection, the tube voltage value for X-ray tube 311, and the target CT value.

The main injection setting screen is a screen set to receive the input of the abovementioned data by the operator. However, when the maximum CT value measured by X-ray CT apparatus 300 in the test injection 300 can be obtained from X-ray CT apparatus 300 through interface 147, the maximum CT value is automatically input from X-ray CT apparatus 300 to injection amount computing section 146 through interface 147, and the input by the operator is omitted. The target CT value may be input as a specific numeric value or may be selected by the operator from several modes preset in control section 141 in accordance with the imaging object or the like.

FIG. 6 shows an example of the main injection setting screen. Main injection setting screen 160 shown in FIG. 6 is a screen when the operator inputs the maximum CT value obtained in the test injection, and not only displays the imaging region set in the test injection but also has a box for inputting the maximum CT value in the test injection, a box for inputting the tube voltage value for X-ray tube 311, and a plurality of buttons for setting the mode of the main injection. The main injection has three modes of a standard mode, a reduced-amount mode, and an expectation mode, and the operator touches one of the buttons as appropriate to select the associated mode. The selected mode is visually distinguished from the other modes. FIG. 6 shows the state in which the reduced-amount mode is selected.

The standard mode is a mode for capturing images with sufficient clarity in all parts. The reduced-amount mode is a mode for capturing images which do not require clarity in all parts and only allow the recognition of the outline. The expectation mode is the mode for capturing images by specifically designating the maximum CT value. For example, in diagnosis of the coronary artery of the heart, the maximum CT value in the standard mode is set to approximately 350 HU so as to perform a diagnosis of coronary artery stenosis and plaque appropriately, and the maximum CT value in the reduced-amount mode is set to approximately 250 HU so as to perform a diagnosis of coronary artery stenosis. The maximum CT values are set in a table or the like held by control section 141 in association with the imaging regions and the modes. When the operator selects the mode, control section 141 sends the data of the maximum CT value associated with the mode to injection amount computing section 146.

While FIG. 6 shows that a specific numeric value should be input for the tube voltage value, there are several tube voltages actually at which the contrast medium is highly responsive (for example 80 kV, 100 kV, and 120 kV, depending on the contrast medium). The main injection setting screen may also be configured such that the operator can select one of those values.

Main injection setting screen 160 has a start button. When the operator touches the start button after the input of the conditions for calculating the amount of contrast medium to be injected in the main injection, control section 141 sends the maximum CT value in the test injection, the tube voltage value, and the target CT value as data to injection amount computing section 146. Injection amount computing section 146 uses the sent data to calculate the amount of contrast medium to be injected in the main injection.

When the data of the tube voltage value can be sent from chemical liquid injector 100 to X-ray CT apparatus 300 through interfaces 147 and 360, the operation of the start button causes control section 141 of chemical liquid injector 100 to transmit the input tube voltage value to X-ray CT apparatus 300. X-ray CT apparatus 300 changes the tube voltage to be applied to X-ray tube 311 in the main injection based on the transmitted tube voltage value. When chemical liquid injector 100 is configured not to transmit the tube voltage value to X-ray CT apparatus 300, the operator sets the tube voltage for X-ray CT apparatus 300 in accordance with the object of the diagnosis prior to the operation of the start button.

In main injection setting screen 160 shown in FIG. 6, a box for inputting the maximum CT value attained time is displayed in addition to the conditions for calculating the amount of contrast medium to be injected. The imaging of X-ray CT apparatus 300 is preferably performed within the period including the maximum CT value attained time. The maximum CT value attained time is used in determining the imaging timing after the start of the injection of the contrast medium.

However, the injected amount of contrast medium and the maximum CT value attained time have a correlation, and typically, as the injected amount of contrast medium is increased, the time to reach the maximum CT value is increased. Thus, the main injection tends to require the longer maximum CT value attained time than in the test injection. When the imaging timing in the main injection is determined by using the maximum CT value attained time obtained in the test injection, the imaging timing in the main injection may be too early to provide intended images.

To address this, control section 141 predicts the maximum CT value attained time in the main injection from the ratio between the amount of contrast medium in the test injection and the amount of contrast medium in the main injection determined by injection amount computing section 146, and in accordance with the predicted value, corrects the transmission timing of the injection start signal to X-ray CT apparatus 300. When X-ray CT apparatus 300 has the sufficient imaging period, it is unnecessary to input the maximum CT value attained time and to correct the transmission timing of the injection start signal in association therewith.

When injection amount computing section 146 calculates the amount of contrast medium to be injected in the main injection, it transmits the calculated value as data to control section 141. Control section 141 controls the operation of two piston driving mechanisms 113 so as to inject the amount of contrast medium calculated by injection amount computing section 146 and thus the main injection is performed.

An injection protocol of contrast medium in the main injection may be different from the injection protocol in the test injection but is preferably the same protocol as that in the test injection. When the main injection is performed with the same protocol as in the test injection, the change characteristics of the CT value over time in the main injection are similar to those in the test injection and thus the result obtained in the test injection can be reflected favorably in the main injection. In addition, in the present embodiment, a predetermined amount of physiological saline is injected at a preset injection speed after the injection of the contrast medium.

Simultaneously with the start of the main injection, a main injection start signal is transmitted from control section 141 to X-ray CT apparatus 300 through interface 147. CT apparatus 300 performs main scan a predetermined time after the start of the main injection. Through the main scan, the CT images of the patient are obtained at the target imaging region. When a deviation of the maximum CT value attained time is predicted on the basis of the maximum CT value attained time determined in the test injection, control section 141 transmits the main injection start signal to X-ray CT apparatus 300 the deviation time after the start of the main injection.

As described above, according to the present embodiment, the amount of contrast medium in the main injection is predicted from the maximum CT value measured in the test injection, the tube voltage value for X-ray tube 311, and the target CT value, so that the optimal amount of contrast medium in view of individual differences can be determined simply. In addition, the tube voltage value applied to X-ray tube 311 is changed to a lower value than usual in accordance with the object of diagnosis, and based on the changed tube voltage value, the amount of contrast medium to be injected in the main injection is determined so as to achieve the target CT value, thereby making it possible to capture the image having information necessary for the diagnosis with a smaller amount of contrast medium. Since the test injection is the step normally performed prior to the main injection to measure the time taken for the contrast medium to reach the imaging region, the conduction of the test injection does not increase an additional step.

While the present embodiment has shown the example in which the maximum CT value obtained in the test injection and the tube voltage value for X-ray tube 311 are used in order to determine the amount of contrast medium in the main injection, data about the symptom of the patient can be additionally used. In this case, input device 142 of chemical liquid injector 100 receives input of the maximum CT value obtained in the test injection, the tube voltage value for X-ray tube 311, and the data about the symptom of the patient, and injection amount computing section 146 calculates the amount of contrast medium to be injected in the main injection in accordance with a calculation expression in which those data items are used as parameters.

For example, when diagnostic images of a patient having symptoms of chronic heart failure or chronic renal failure are obtained, the CT value tends to be lower than the other patients, so that intended images may not be taken even when that patient is injected with the same amount of contrast medium as that in the other patients. Thus, the amount of contrast medium is determined in view of the symptom of the patient as described above to allow more favorable images to be taken.

When the amount of contrast medium is determined in view of the symptom of the patient as described above, however, the injected amount of contrast medium is increased as compared with the other case to increase the burden on the patient's body. Thus, control section 141 of chemical liquid injector 100 preferably displays on display device 143 the amount of contrast medium calculated in injection amount computing section 146 together with an alarm text, for example “This symptom requires a contrast medium of XX ml or more. The operation should be continued?” to call attention of the operator. The display of the amount of contrast medium computed by injection amount computing section 146 on display device 143 is applicable not only to the case where the symptom of the patient is considered but also to the case where the amount of contrast medium is calculated from the maximum CT value in the test injection and the tube voltage value for X-ray tube 311.

While the abovementioned embodiment has shown the example in which two chemical liquid syringes 200P and 200C are mounted on injection head 110, the number of the chemical liquid syringes mountable on injection head 110 can be one or three or more. In this case, a plurality of concave portions 114, piston driving mechanisms 130 and the like are provided in accordance with the number of the chemical liquid syringes to be mounted. The chemical liquid syringe mountable on injection head 110 has an arbitrary size, and the size of concave portion 114, the stroke of rod 131 and the like are set so as to adapt to the size of the chemical liquid syringe which should be mounted. When a plurality of chemical liquid syringes are mounted, these chemical liquid syringes may have different sizes.

### DESCRIPTION OF REFERENCE NUMERALS

100 CHEMICAL LIQUID INJECTOR
101 INJECTION CONTROL UNIT
110 INJECTION HEAD
113 PISTON DRIVING MECHANISM
141 CONTROL SECTION
142 INPUT DEVICE
143 DISPLAY DEVICE
146 INJECTION AMOUNT COMPUTING SECTION
147 INTERFACE
200C, 200P SYRINGE
300 X-RAY CT APPARATUS
310 IMAGING UNIT
311 X-RAY TUBE
312 X-RAY DETECTOR
320 BED UNIT
330 CONTROL SECTION
340 INPUT DEVICE
350 DISPLAY DEVICE
360 INTERFACE
1000 X-RAY CT SYSTEM

1. A chemical liquid injector comprising:
an injection head injecting a contrast medium into a patient through operation of a syringe filled with the contrast medium,
a control section controlling operation of the injection head so as to perform test injection prior to main injection, for obtaining CT images of the patient with an X-ray CT apparatus,
input means for receiving input of data including a maximum CT value measured by the X-ray CT apparatus in the test injection, a tube voltage value to be applied to an X-ray tube of the X-ray CT apparatus in the main injection, and a target CT value serving as a targeted CT value in the main injection; and
an injection amount computing section calculating an amount of contrast medium to be injected in the main injection based on the maximum CT value and the tube voltage value such that a maximum CT value measured in the main injection is the target CT value,
wherein the control section controls the injection head in the main injection such that the amount of contrast medium calculated in the injection amount computing section is injected from the syringe.

2. The chemical liquid injector according to claim 1, wherein the input means receives input of the data further including a maximum CT value attained time which is a time from start of injection of the contrast medium to attainment of the maximum CT value, the maximum CT value attained time being measured by the X-ray CT apparatus in the test injection, and
the control section corrects a transmission timing of an injection start signal to the X-ray CT apparatus in accordance with a predicted value of the maximum CT attained time in the main injection determined on the basis of the amount of contrast medium calculated in the injection amount computing section.

3. The chemical liquid injector according to claim 1 or 2, wherein the input means is configured to receive selection from a plurality of modes in which the target CT values are different, in the input of the target CT value, and
the control section transmits data of the target CT value in accordance with the mode selected in the input means to the injection amount computing section.

4. The chemical liquid injector according to claim 1, wherein the input means is an input device receiving input operation of the maximum CT value, the tube voltage, and the target CT value by an operator.

5. The chemical liquid injector according to claim 1, wherein the input means is an input device receiving input operation of the tube voltage and the target CT value by an operator and an interface receiving the maximum CT value measured in the X-ray CT apparatus from the X-ray CT apparatus.

6. The chemical liquid injector according to claim 1, further comprising a display device,
wherein the control section causes the display device to display the amount of contrast medium measured in the injection amount computing section.

7. An X-ray CT system comprising:
the chemical liquid injector according to claim 1, and an X-ray CT apparatus,
wherein each of the chemical liquid injector and the X-ray CT apparatus has an interface, the interfaces being capable of transmission and reception of data therebetween, and
the maximum CT value measured by the X-ray CT apparatus in the test injection is transmitted to the injection amount computing section through the interface.

8. The X-ray CT system according to claim 7, wherein the tube voltage value input from the input means of the chemical liquid injector is transmitted to the X-ray CT apparatus through the interface, and the X-ray CT value changes the tube voltage to be applied to the X-ray tube in the main injection in accordance with the tube voltage value transmitted from the chemical liquid injector.
